(19) United States
(12) Patent Application Publication
(10) Pub. No.: US 2003/0180373 A1
(43) Pub. Date: Sep. 25, 2003

(54) Method for viral inactivation of hydro-alcoholic solutions

(76) Inventor: Anne-Marie Binsard, Neuilly-Sur-Seine (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/240,940

(22) PCT Filed: Apr. 3, 2001
(86) PCT No.: PCT/FR01/00988

(30) Foreign Application Priority Data
Apr. 3, 2000 (FR) 00/04212

(51) Int. Cl. A61K 35/78; A61K 33/00

(52) U.S. Cl. 424/600; 424/725

(57) ABSTRACT

The invention concerns a method for viral inactivation of any hydro-alcohol solution and in particular any homeopathic dilution produced in a hydro-alcoholic medium, characterised in that it consists in heating the solution at a temperature ranging between 30° C. and 65° C. ? 1° C., for one hour, in the presence of ethanol with alcohol content ranging between 10% v/v and 75% v/v.

The present invention relates to a process for the viral inactivation of any aqueous-alcoholic solution and especially homeopathic dilutions.

In homeopathy the therapeutic substances usually used are more or less dilute solutions, which are the active principles of the medicinal products. Homeopathic dilutions are usually made by the Hahnemann method. They are carried out in an aqueous-alcohol mixture of suitable titer. It is necessary when working with them to avoid any soiling or contamination of chemical or particulate nature, given the tiny doses of active principle usually involved.

Among the raw materials used in homeopathy, use is made of substances of animal origin (snake venom, bee venom) and/or of biological origin, which are liable to be contaminated with viruses that are potentially pathogenic and hazardous to patients.

Viral infections are a major problem of public health; they are associated with the ingestion of contaminated food or drink, with the environment or with contact between individuals. Many viral inactivation processes have been used to combat such infections.

Thus, in the context of public-sector premises (hospitals, schools, retirement homes), in order to avoid gastroenteritis epidemics caused by viruses of Norwalk and Norwalk type, Doultree J. C. et al. (Journal of Hospital Infection, 1999, 41, 51-57) have shown that, among the disinfectants conventionally used, glutaraldehyde, iodine derivatives and bleach are capable of destroying the virus, whereas quaternary ammonium derivatives, detergents and ethanol at a concentration of 75% have no effect. These authors have also shown that the total inactivation of the virus is obtained only after heating for one hour at 56° C. and after 5 minutes at a temperature of 70° C.

In the case of blood derivatives of human or animal origin, which are used for therapeutic, prophylactic or diagnostic purposes in man and which present a high risk of infection to patients (AIDS, hepatitis-C, etc.), viral inactivation is fundamental.

However, in the case of these derivatives, the viral inactivation methods used must make it possible to inactivate the viruses that are potentially hazardous to the patient while at the same time conserving the biological properties of the blood derivatives, and such processes are always difficult to carry out.

Furthermore, since viruses are not all sensitive to the same agents, it is usually necessary to combine various inactivation techniques, which complicates the implementation of the processes.

Among the various inactivation methods used in this context, mention may be made especially of heating blood derivatives in aqueous solution or in dry form, treatment of these derivatives with detergents and solvents, optionally followed by heating of the product in dry form.

Given the need to ensure viral inactivation in preparations for homeopathic use, the development of novel adapted, fast and economic processes for inactivating a large number of viruses is found to be necessary.

Now, the inventors have found, surprisingly, that by moderately heating homeopathic dilutions prepared from a mixture of water and ethanol that contain a viral overload, at temperatures that are compatible with their therapeutic activity, inactivation of the virus is obtained, owing to a synergistic action between the ethanol present in these dilutions and the heating maintained for a sufficient period.

Thus, one subject of the present invention is a process for the viral inactivation of any aqueous-alcoholic solution and in particular homeopathic dilutions, characterized in that it includes a step of heating at a temperature of between 39° C. and 65° C.±1° C., for at least one hour, in the presence of ethanol with an alcoholic titer of between 10% v/v and 75% v/v.

In one advantageous embodiment of the invention, the homeopathic dilutions are chosen from the group consisting of Hahnemann dilutions, Korsakow dilutions and Millesimal dilutions.

In another advantageous embodiment of the invention, the heating time is between at least 1 hour and 24 hours.

In one even more advantageous embodiment of the invention, the temperature is between 39° C. and 54° C.±1° C.

The process according to the invention is easy to carry out and allows the inactivation of viruses that are particularly resistant to any physicochemical treatment, especially viruses of the Parvoviridae family, of the genus Parvovirus, for instance minute virus of mice (MVM).

Examples 1 and 2 below illustrate the invention without, however, limiting it.

### EXAMPLE 1

1.1 Materials and Method

1.1.1. Homeopathic Dilution

Snake venom at the 3 CH dilution (3rd Centesimal Hahnemann Dilution) prepared in 40% ethanol (v/v) was used as starting material. It is referred to as Lachesis 3CH.

1.1.2. Minute Virus of Mice (MVM)

Minute virus of mice belongs to the Parvovididae family, genus Parvovirus. It is a nonenveloped virus, of icosahedral symmetry, 18 to 26 nm in diameter. The genome is a 5-kb negative-polarity single-stranded DNA molecule.

Parvoviruses are highly diversified specific species, but some of them cross the species barrier, and they furthermore have exceptional physicochemical properties.

The majority of Parvoviruses are contaminants of rodents (rats, mice, hamsters) and of carnivores (cats, dogs, etc.).

The tissues bearing Parvoviruses are mainly: the kidneys, the pancreas, the lungs, lymphatic tissues, bone marrow, the testicles. They can give many and varied infections: diarrhea, enteritis, hepatitis, etc.

These viruses show great resistance to high temperatures (60 to 80° C.), to dehydration, to pH variations and to solvent/detergent treatments.

Thus, it is difficult to inactivate or eliminate these viruses in products during or after the pharmaceutical production process.

MVM (prototype strain) is obtained from the culture supernatant of infected A9 cells. After clarification at 3 500 rpm (Sigma 3K2) for 15 minutes, the cells are collected and resuspended in PBS buffer (8.0 g/l NaCl, 1.44 g/l Na₂HPO₄.2H₂O, 0.2 g/l KCl, 0.2 g/l KH₂PO₄, pH=4), sonicated, clarified at 2 500 rpm for 10 minutes, divided into aliquots and frozen at −75° C.±5° C. The supernatants are concentrated by ultracentrifugation at 1 900 rpm for 15 hours (Beckman R19 rotor). The pellet is resuspended in PBS buffer, divided into aliquots and frozen at −75° C.±5° C. The titer is determined by the agarose plaque method on A9 cells. The viral stock used has a titer of 5.2×10⁷ plaque-forming units per milliliter (PFU/ml).

1.1.3. Cells

These are A9 cells (Deutsches Krebsforschungzentrum, Heidelberg) obtained from a continuous line of mouse fibroblasts. These cells are cultured in DMEM containing 4.5 g/l of glucose, 110 mg/l of sodium pyruvate and 1.1 g/l of sodium bicarbonate, supplemented with 4 mM of glutamine and 5% fetal calf serum.

1.1.4. Preparation of Samples Containing a Viral Overload

100 ml of starting material overloaded with MVM to a final concentration of 1.72×10⁶ plaque-forming units per milliliter (PFU/ml) were prepared at room temperature (16° C.-26° C.).

1.1.5. Incubation and Titration

After homogenization, a sample was taken for the positive controls L1, L2 and L3. The overloaded starting material was divided into two portions of 45 ml each. Each portion was then incubated either at 39° C.±1° C., or at 54° C.±1° C., without agitation, for 8 hours.

Samples were taken after 1 hour, 3 hours and 8 hours of incubation at a rate of two samples at a time (A and B). These were immediately diluted in culture medium and ultracentrifuged at 95 000 rpm for 20 minutes at about 4° C. (Beckman TLA 100.4 rotor). The pellet was resuspended in the culture medium, divided into aliquots and frozen at −75° C.±5° C. until the moment of titration.

The time zero of the experiment was taken from the moment the temperature of the overloaded starting material reached the lower limit of the test temperature.

The temperature of the experiments was controlled and recorded using calibrated thermometers or probes.

The mean reduction factor, noted m(R), is obtained by calculating the difference between the logarithms of the mean titers of each sample concerned.

1.1.6. Controls

V1: Stock of virus diluted in culture medium, divided into aliquots and frozen at −75° C.±5° C. until the moment of titration.

V2: Stock of virus diluted in culture medium and ultracentrifuged as described above. The pellet will be resuspended in the culture medium, divided into aliquots and frozen at −75° C.±5° C. until the moment of titration.

V3: Stock of virus diluted in culture medium, left at room temperature (16° C.-26° C.) for the duration of the experiment, and then ultracentrifuged as described above. The pellet will be resuspended in the culture medium, divided into aliquots and frozen at −75° C.±5° C. until the moment of titration.

L1: Overloaded starting material diluted 50-fold in culture medium, divided into aliquots and frozen at −75° C.±5° C. until the moment of titration.

L2: Overloaded starting material diluted in culture medium and ultracentrifuged as described above. The pellet will be resuspended in the culture medium, divided into aliquots and frozen at −75° C.±5° C. until the moment of titration.

L3: Overloaded starting material, left at room temperature (16° C.-26° C.) for the duration of the experiment and then ultracentrifuged as described above. The pellet will be resuspended in the culture medium, divided into aliquots and frozen at −75° C.±5° C. until the moment of titration.

1.2. Results

1.2.1. Controls Without Heating

The results are collated in table 1 and expressed as plaque-forming units per milliliter (PFU/ml).

Snake venom at the 3CH dilution (3rd Hahnemann Centesimal Dilution) prepared in 40% v/v ethanol and ultracentrifuged showed no toxicity to the cells of the undiluted MVM titration (negative control N2).

No significant loss of infectious particles was observed during the ultracentrifugation of the overloaded MVM, either in the culture medium (V2 compared with V1), or in the starting material (L2 compared with L1).

No significant loss of infectious particles was observed during the incubation at the laboratory temperature for the duration of the experiment of the overloaded MVM, either in the culture medium (V3 compared with V2), or in the starting material (L3 compared with L2).

1.2.2. Effect of Heating in the Presence of Ethanol

The results are collated in table 2 and expressed as plaque-forming units per milliliter (PFU/ml).

The results obtained show that heating at 39° C.±1° C. in the presence of 40% ethanol (v/v) cannot inactivate the MVM, even after incubation for 8 hours.

On the other hand, heating at 54° C.±1° C. under the same conditions produced a gradual inactivation of the MVM virus over time to reach 2.77 log (sample A) and 2.84 log (sample B) after 8 hours.

TABLE 1
CONTROLS

| | | Infectious titer | | |
|---|---|---|---|---|
| | | (T) | | |
| | | Mean titer [m(T)] | Calculation of the reduction factor ® | |
| Description of the samples | | and confidence | | Confidence |
| Code | Definition | interval (PFU/ml) | Mean reduction factor m (R) | interval |
| N2 | Negative control: | Nontoxic | | |
| | Snake venom at the 3 CH dilution in 40% | | | |
| | (v/v) ethanol (starting material), | | | |
| | ultracentrifuged culture medium | | | |
| V1 | Positive controls: | | Viral stock control | |
| | Stock of virus diluted in culture medium | | | |
| | Experiments A and B | (6.70 ± 1.43)10⁵(a) | | |
| V2 | Stock of virus diluted in culture | | Effect of the | 0.07 ≦ R ≦ 0.52 |
| | medium and then | | ultracentrifugation on the | |
| | ultracentrifuged | | virus in culture medium | |
| | Experiment A and Experiment B | (3.46 ± 1.02)10⁵(b) | log (a) − log (b) = 0.29 | |
| V3 | Stock of virus diluted in culture medium, | | Effect of the duration of the | |
| | left at room temperature for the duration | | experiment on the virus in | |
| | of the experiment and then ultracentrifuged | | culture medium | |
| | Experiment A and Experiment B | (7.08 ± 1.46)10⁵(c) | log b −log (c) ≈ 0 | |
| L1 | Stock of virus diluted in the starting | | Effect of the starting material | |
| | material and diluted in culture medium | | on the virus | |
| | Experiment A | (1.44 ± 0.30)10⁶(d1) | log (a) − log (d1) ≈ 0 | |
| | Experiment B | (9.40 ± 2.40)10⁵(d2) | log (a) − log (d2) ≈ 0 | |
| L2 | Stock of virus diluted in | | Effect of the | |
| | the starting material and diluted | | ultracentrifugation on the | |
| | in culture medium | | virus in the starting material | |
| | Experiment A | (2.66 ± 0.80)10⁵(e1) | log (d1) − log (e1) = 0.73 | 0.52 ≦ R ≦ 0.97 |
| | Experiment B | (2.98 ± 0.86)10⁵(e2) | log (d2) − log (e2) = 0.50 | 0.26 ≦ R ≦ 0.75 |
| L3 | Stock of virus diluted in the starting | | Effect of the duration of the | |
| | material, left at room temperature for 8 | | experiment on the virus in the | |
| | hours and then ultracentrifuged | | starting material | |
| | Experiment A | (4.32 ± 1.02)10⁵(f1) | log (e1) − log (f1) ≈ 0 | |
| | Experiment B | (6.50 ± 1.26)10⁵(f2) | log (e2) − log (f2) ≈ 0 | |

TABLE 2

| Sample | | | Infectious titer (PFU/ml) | |
|---|---|---|---|---|
| Stock of virus diluted in | | | (3.46 + 1.02)10⁵ | |
| culture medium and | | | | |
| ultracentrifuged (PFU/ml) | | | | |
| Stock of virus diluted in the | | | | |
| starting material and | | | | |
| ultracentrifuged | | | | |
| | | Experiment A | (2.66 ± 0.80)10⁵ | |
| | | Experiment B | (2.98 ± 0.86)10⁵ | |
| Stock of virus diluted in the | | | 39° C. ± 1° C. | 54° C. ± 1° C. |
| starting material incubated at | | | | |
| for: | 1 hour | Experiment A | results not | (8.46 ± 1.42)10⁵ |
| | | | recorded | |
| | | Experiment B | (2.46 ± 0.38)10⁵ | (8.84 ± 2.32)10⁴ |
| | 3 hours | Experiment A | (1.10 ± 0.16)10⁵ | (5.76 ± 1.50)10³ |
| | | Experiment B | (1.55 ± 0.39)10⁵ | results not |
| | | | | recorded |
| | 8 hours | Experiment A | (3.82 ± 0.96)10⁴ | (4.54 ± 1.06)10² |
| | | Experiment B | (8.88 ± 3.00)10⁴ | (4.30 ± 1.02)10² |

| Mean reduction factor m(R) | | | |
|---|---|---|---|
| and confidence interval | | | |
| (Rmin ≦ R ≦ Rmax) | | | |
| after heating for 8 hours | | | |
| in the presence of 40% ethanol | | | |
| (v/v) | | | |
| | Experiment A | 0.84 | 2.77 |
| | | 0.59 ≦ R ≦ 1.08 | 2.52 ≦ R ≦ 3.00 |
| | Experiment B | 0.53 | 2.84 |
| | | 0.25≦ R ≦ 0.81 | 2.60 ≦ R ≦ 3.07 |

### EXAMPLE 2

2.1. Materials and Method

2.2.1. Homeopathic Dilution

Snake venom at the 3DH dilution (3rd Decimal Hahnemann Dilution) prepared in 40% v/v ethanol was used as starting material and referred as Lachesis 3DH (batch No. PA 482).

2.1.2. Minute Virus of Mice (MVM)

This is obtained according to the procedure described in 1.1.2.

2.1.3. Cells

The cells are cultured according to the procedure described in 1.1.3.

2.1.4. Preparation of Samples Containing a Viral Overload

The experiment was performed in duplicate (experiments A and B) at 54° C.±1° C. over 6 contact times with a 3DH dilution (3rd Decimal Hahnemann Dilution) prepared in 40% v/v ethanol of the Lachesis strain.

Two independent overloads were prepared at room temperature (16° C.-26° C.) with the MVM virus, so as to have a final MVM concentration of 2.26×10⁷ PFU/ml.

The first overload was used for the samples taken at times 1 hour, 3 hours, 8 hours and 10 hours.

The second overload was used for the samples taken at times 1 hour, 16 hours and 20 hours.

2.1.5. Incubation and Titration

The overloaded starting material was incubated at 54° C.±1° C., without agitation, for 20 hours.

The time zero of the experiment started when the temperature of overloaded starting material reached the lower limit of the test temperature.

Samples were taken after 1 hour, 3 hours, 8 hours, 10 hours, 16 hours and 20 hours of incubation. These samples were immediately cooled, diluted in culture medium and ultracentrifuged at 95 000 rpm for 20 minutes at about 4° C. (Beckman TLA 100.4 rotor). The pellets were resuspended in the culture medium, divided into aliquots and frozen at −75° C.±5° C. until the moment of titration.

The temperature of the experiments was controlled and recorded using calibrated thermometers or probes.

The mean reduction factor, noted m(R), is obtained by calculating the difference between the logarithms of the mean titers of each sample concerned.

2.1.6. Controls

V1: Stock of virus diluted in culture medium, divided into aliquots and frozen at −75° C.±5° C. until the moment of titration.

V2: Stock of virus diluted in culture medium and ultracentrifuged as described above. The pellet was resuspended in the culture medium, divided into aliquots and frozen at −75° C.±5° C. until the moment of titration.

V3: Stock of virus diluted in culture medium, left at room temperature (16° C.-26° C.) for the duration of the experiment, and then ultracentrifuged as described above. The pellet was resuspended in the culture medium, divided into aliquots and frozen at −75° C.±5° C. until the moment of titration.

V4: Stock of virus diluted in the culture medium, heated at 54° C.±1° C. for 16 hours (V4 a) and 20 hours (V4 b), and then ultracentrifuged as described above. The pellet was resuspended in the culture medium, divided into aliquots and frozen at −75° C.±5° C. until the moment of titration.

L1: Overloaded starting material diluted 50-fold in culture medium, divided into aliquots and frozen at −75° C.±5° C. until the moment of titration.

L2: Overloaded starting material diluted in culture medium and ultracentrifuged as described above. The pellet was resuspended in the culture medium, divided into aliquots and frozen at −75° C.±5° C. until the moment of titration.

L3: Overloaded starting material (2nd overload) left at room temperature for 16 hours (L3 a) and 20 hours (L3 b) and then ultracentrifuged as described above. The pellet was resuspended in the culture medium, divided into aliquots and frozen at −75° C.±5° C. until the moment of titration.

2.2. Results

2.2.1 Controls

*Effect of the Starting Material on the Viral Infectivity

No significant loss of infectious particles was observed when the virus was diluted in the starting material, Lachesis 3DH, (3rd Decimal Hahnemann Dilution) prepared in 40% v/v ethanol and then stored at −75° C.±5° C. until the moment of titration [L1 (3.16±0.44)×10⁷ compared with VI (3.64±0.67)×10⁷].

*Effect of Ultracentrifugation on the Viral Infectivity

No significant loss of infectious particles was observed for the MVM virus diluted in culture medium [V2 (3.64±0.67)×10⁷ compared with V1].

On the other hand, a significant loss of viral particles was observed when the virus diluted in the starting material was ultracentrifuged.

This loss of infectious particles is probably due to the starting material rather than to the ultracentrifugation conditions used. However, this loss of infectious particles will not be taken into account in the calculation of the reduction factor for the step.

* Effect of Heating the Virus in Culture Medium at 54° C.±1° C.

No significant loss of infectious particles was observed after 20 hours of heating at 54° C.±1° C. of the culture medium overloaded with MVM [V4 a (8.44±1.44)×10⁶ compared with V2] and [V4 b (9.46±1.52)×10⁶ compared with V2].

* Effect of the Duration of the Experiment on the Infectivity of the Virus Diluted in Starting Material

No significant loss of infectious particles was observed after incubation for 16 hours and 20 hours at room temperature of the overloaded starting material [L3 a (3.04±0.96)×10⁶ compared with L2 (1.13±0.17)×10⁶] and [L3 b (1.60±0.31)×10⁶ compared with L2].

2.2.2. Effect of Heating in the Presence of Ethanol

The results are collated in tables 3.A and 3.B and expressed as plaque-forming units per milliliter (PFU/ml).

Heating at 54° C.±1° C. of the 3DH dilution (3rd Decimal Hahnemann Dilution) performed in 40% v/v ethanol of the Lachesis strain produces a very significant inactivation of the MVM after 20 hours.

Specifically, a loss of 2.90 log (Experiment A) and of 2.80 log (Experiment B) was obtained.

Similar results were obtained for the 2 kinetics performed.

TABLE 3.A
EXPERIMENT A

| | | Infectious titer (T) | | |
|---|---|---|---|---|
| | | Mean titer [m(T)] and | Evaluation of the reduction factor (R) | |
| Sample | | confidence interval | | Confidence |
| Definition | | (PFU/ml) | Mean reduction factor m(R) | interval |
| Stock of virus diluted in | | | | |
| the starting material* | | | | |
| 1st overload | | (1.13 ± 0.17)10⁶ (f1) | | |
| 2nd overload | | (1.48 ± 0.30)10⁶ (f3) | | |
| and then ultracentrifuged | | | | |
| Stock of virus diluted in | | | | |
| the starting material*, | | | | |
| incubated at 54° C. ± 1° C. | | | | |
| without agitation, for: | | | | |
| 1st overload | 1 hour | (4.48 ± 0.52)10⁵ (h1) | log(f1) − log(h1) = 0.40 | 0.29 ≦ R ≦ 0.52 |
| 2nd overload | | (1.01 ± 0.52)10⁶ (H3) | log(f3) − log(h3) ≈ 0 | |
| 1st overload | 3 hours | (2.56 ± 0.25)10⁵ (i1) | log(f1) − log(i1) = 0.65 | 0.51 ≦ R ≦ 0.78 |
| 1st overload | 8 hours | (5.46 ± 1.16)10⁴ (j1) | log(f1) − log(j1) = 1.32 | 1.17 ≦ R ≦ 1.48 |
| 1st overload | 10 hours | (1.69 ± 0.32)10⁴ (k1) | log(fl) − log(k1) = 1.83 | 1.68 ≦ R ≦ 1.98 |
| 2nd overload | 16 hours | (1.25 ± 0.28)10⁴ (l3) | log(f3) − log(l3) = 2.07 | 1.89 ≦ R ≦ 2.26 |
| 2nd overload | 20 hours | (1.86 ± 0.32)10³ (m3) | log(f3) − log(m3) = 2.90 | 2.79 ≦ R ≦ 2.99 |

| | | |
|---|---|---|
| and then ultracentrifuged | | |
| Reduction factor obtained | 2.90 | 2.79 ≦ R ≦ 2.99 |
| for the longest time | | |

TABLE 3.B
EXPERIMENT B

| | | Infectious titer (T) | | |
|---|---|---|---|---|
| | | Mean titer [m(T)] and | Evaluation of the reduction factor (R) | |
| Sample | | confidence interval | | Confidence |
| Definition | | (PFU/ml) | Mean reduction factor m(R) | interval |
| Stock of virus diluted in | | | | |
| the starting material | | | | |
| 1st overload | | (2.26 ± 0.38)10⁶ (f2) | | |
| 2nd overload | | (2.16 ± 0.36)10⁶ (f4) | | |
| and then ultracentrifuged | | | | |
| Stock of virus diluted in | | | | |
| the starting material*, | | | | |
| incubated at 54° C. ± 1° C. | | | | |
| without agitation, for: | | | | |
| 1st overload | 1 hour | (6.80 ± 1.28)10⁵ (h2) | log(f2) − log(h2) = 0.52 | 0.37 ≦ R ≦ 0.68 |
| 2nd overload | | (8.68 ± 2.30)10⁵ (H4) | log(f4) − log(h4) = 0.40 | 0.21 ≦ R ≦ 0.60 |
| 1st overload | 3 hours | (1.92 ± 0.34)10⁵ (i2) | log(f2) − log(i2) = 1.07 | 0.92 ≦ R ≦ 1.22 |
| 1st overload | 8 hours | (2.30 ± 0.74)10⁴ (j2) | log(f2) − log(j2) = 1.99 | 1.79 ≦ R ≦ 2.23 |
| 1st overload | 10 hours | (2.00 ± 0.70)10⁴ (k2) | log(f2) − log(k2) = 2.05 | 1.84 ≦ R ≦ 2.31 |
| 2nd overload | 16 hours | (1.25 ± 0.28)10⁴ (l4) | log(f4) − log(l4) = 2.24 | 2.07 ≦ R ≦ 2.41 |
| 2nd overload | 20 hours | (3.44 ± 0.94)10³ (m4) | log(f4) − log(m4) = 2.80 | 2.61 ≦ R ≦ 3.00 |

| | | |
|---|---|---|
| and then ultracentrifuged | | |
| Reduction factor obtained for the longest time | 2.80 | 2.79 ≦ R ≦ 2.99 |

1. A process for the viral inactivation of any aqueous-alcoholic solution and in particular any homeopathic dilution in aqueous-alcoholic medium, characterized in that it includes a step of heating at a temperature of between 39° C. and 65° C.±1° C., for at least one hour, in the presence of ethanol with an alcoholic titer of between 10% v/v and 75% v/v.

2. The process as claimed in claim 1, characterized in that the homeopathic dilution is chosen from the group consisting of Hahnemann dilutions, Korsakow dilutions and Millesimal dilutions.

3. The process as claimed in either of claims 1 and 2, characterized in that the heating time is between at least 1 and 24 hours.

4. The process as claimed in any one of claims 1 to 3, characterized in that the temperature is between 39° C. and 54° C.±1° C.
